(19) United States
(12) Patent Application Publication
(10) Pub. No.: US 2005/0278871 A1
(43) Pub. Date: Dec. 22, 2005

(54) Compositions comprising at least one compound chosen from heptafulvene and tropolone derivatives, and dyeing process thereof

(76) Inventors: Luc Gourlaouen, Asnieres (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/980,278

(22) Filed: Nov. 4, 2004

Related U.S. Application Data
(60) Provisional application No. 60/518,283, filed on Nov. 10, 2003.

(30) Foreign Application Priority Data
Nov. 5, 2003 (FR) 03 50788

(51) Int. Cl. A61K 8/00

(52) U.S. Cl. 8/405

(57) ABSTRACT

The present disclosure relates to compositions comprising, in a medium that is suitable for dyeing keratin fibers, such as human keratin fibers, at least one compound chosen from heptafulvene and tropolone derivatives and mixtures thereof. The present disclosure similarly relates to a process for dyeing keratin fibers using the compositions, optionally in the presence of at least one oxidizing agent, and also to a multi-compartment kit comprising at least one compartment comprising a composition as disclosed herein, and at least one compartment comprising a composition comprising at least one oxidizing agent.

This application claims benefit of U.S. Provisional Application No. 60/518,283, filed Nov. 10, 2003.

The present disclosure relates to a composition for dyeing keratin fibers, comprising, in a suitable medium, at least one compound chosen from heptafulvene and tropolone derivatives, and also mixtures thereof, and also to dyeing processes and kits using such compositions.

As stated above, the present disclosure relates to the field of dyeing keratin fibers, for instance, human keratin fibers, such as the hair. More specifically, the present disclosure relates to colorations obtained using dye compositions comprising at least one direct dye.

Direct dyes are colored and coloring substances that have a certain affinity for human keratin fibers. Among the direct dyes that are known, non-limiting mention may be made of the class of nitrobenzene dyes or heterocyclic direct dyes, for instance, which can be used to obtain not only natural colors, but also chromatic colors.

Although these dyes have been used extensively in the field of hair dyeing, they may nevertheless pose problems of color uptake in the fiber, for example, with regard to the strength and selectivity obtained. Moreover, the color-fastness of these dyes still remains to be improved.

Thus, one aspect of the present disclosure relates to compositions comprising at least one direct dye, which make it possible to obtain better dyeing results than with benzenic and heterocyclic direct dyes.

Accordingly, a first aspect of the present disclosure is thus a composition comprising, in a medium that is suitable for dyeing keratin fibers, such as human keratin fibers, at least one compound of formula (I): 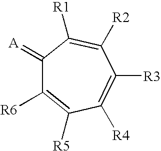
wherein:
R₁ and R₆, which may be identical or different, are chosen from hydrogen atoms; alkyl radicals, aryl radicals, alkylaryl radicals and arylalkyl radicals, in which the alkyl is chosen from C₁-C₆, linear and branched, substituted and unsubstituted alkyl radicals, and the aryl is chosen from C₆-C₁₂, substituted and unsubstituted aryl radicals; halogen atoms; C₁-C₆ alkoxy radicals; amino radicals; amino radicals substituted with at least one radical, which may be identical or different, chosen from linear and branched, substituted and unsubstituted C₁-C₆ alkyl radicals; hydroxyl radicals; —COOX radicals, wherein X is chosen from a hydrogen atom, alkali metals, alkaline-earth metals, and linear and branched, substituted and unsubstituted C₁-C₆ alkyl radicals; an alkylcarbonyl radical wherein the alkyl is chosen from C₁-C₆, linear and branched, substituted and unsubstituted alkyl radicals; —SO₃X′ radicals wherein X′ is chosen from a hydrogen atom, alkali metals and alkaline-earth metals;
R₂, R₃, R₄ and R₅, which may be identical or different, are chosen from hydrogen atoms; linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals; arylalkyl radicals wherein the alkyl is chosen from C₁-C₆, linear and branched, substituted and unsubstituted alkyl radicals, and the aryl is chosen from C₆-C₁₂ aryl radicals; and substituted and unsubstituted cyclic ands polycyclic radicals comprising at least one hetero atom;
A is chosen from oxygen and sulfur atoms, and C(R₇)(R₈) radicals wherein R₇ and R₈, which may be identical or different, are chosen from electron-withdrawing monovalent groups.

The present disclosure furthermore relates to a ready-to-use composition comprising the composition as disclosed herein and at least one oxidizing agent.

Another aspect of the present disclosure is similarly a process for dyeing human keratin fibers using the compositions as disclosed herein, and also a device for performing the process.

Still another aspect of the present disclosure is the use of at least one compound of formula (I) as a direct dye for dyeing keratin fibers, such as human keratin fibers, in order to obtain natural and chromatic shades.

For example, it has been found, unexpectedly, that the presence of at least one compound of formula (I) makes it possible to obtain natural and chromatic shades, which are for instance, strong and sparingly selective. However, other characteristics and advantages of the present disclosure will become evident upon reading the description and the examples that follow.

In the text hereinbelow, the contents of the various compounds present in the compositions are expressed on a weight basis relative to the weight of the composition, unless it is specified that these contents are relative to the weight of the ready-to-use composition. It is noted that the ready-to-use composition comprises the composition according to the present disclosure and at least one oxidizing agent. Moreover, unless otherwise indicated, the limits delimiting a range of values are included within that range.

Finally, the keratin fibers treated in accordance with the present disclosure can be, for example, human keratin fibers such as the hair.

The composition according to the present disclosure comprises at least one compound of formula (I), as mentioned previously.

By way of non-limiting example, in one embodiment of formula (I), R₁ and R₆, which may be identical or different, are chosen from hydrogen atoms; alkyl radicals, aryl radicals, alkylaryl radicals and arylalkyl radicals wherein the alkyl is chosen from C₁-C₆, linear and branched, substituted and unsubstituted alkyl radicals, and the aryl is chosen from substituted and unsubstituted C₆-C₁₂ aryl radicals, such as substituted and unsubstitued C₆ radicals; halogen atoms; C₁-C₆ alkoxy radicals; amino radicals; amino radicals substituted with at least one radical, which may be identical or different, chosen from linear and branched, substituted and unsubstituted C₁-C₆ alkyl radicals; hydroxyl radicals; —COOX radicals, wherein X is chosen from a hydrogen atom, alkali metals alkaline-earth metals, and linear and branched, substituted and unsubstituted C₁-C₆ alkyl radicals; alkylcarbonyl radicals wherein the alkyl is chosen from C₁-C₆, linear and branched, substituted and unsubstituted alkyl radicals; —SO₃X′ radicals wherein X′ is chosen from a hydrogen atom and alkali metals, and alkaline-earth metals, for instance sodium, potassium, magnesium and calcium.

As mentioned previously, in formula (I), R₂, R₃, R₄ and R₅, which may be identical or different, can be chosen from hydrogen atoms and linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals, such as C₆ radicals; arylalkyl radicals in which the alkyl is chosen from C₁-C₆, linear and branched, substituted and unsubstituted alkyl radicals, and the aryl is chosen from C₆-C₁₂, such as C₆, aryl radicals; and substituted and unsubstituted cyclic and polycyclic radicals comprising at least one hetero atom.

Unless otherwise indicated, a radical is said to be substituted if it bears at least one group chosen from:
hydroxyl groups;
halogen atoms, such as chlorine and fluorine;
C₁-C₆ alkoxy groups;
monohydroxyalkoxy groups in which the alkyl is chosen from C₁-C₆, linear and branched, substituted and unsubstituted radicals;
polyhydroxyalkoxy groups, in which the alkyl is chosen from C₂-C₆, linear and branched, substituted and unsubstituted radicals;
amino groups, which can be substituted and unsubstituted with at least one radical chosen from linear and branched, substituted and unsubstituted C₁-C₆ alkyl radicals;
thiol groups;
alkylthio groups in which the alkyl is chosen from C₁-C₆, linear and branched, substituted and unsubstituted radicals;
carboxylic groups in acid and salified form (for example salified with an alkali metal or a substituted or unsubstituted ammonium);
alkylcarbonyl group in which the alkyl is chosen from C₁-C₆, linear and branched, substituted and unsubstituted alkyl radicals;
alkoxycarbonyl groups in which the alkyl is chosen from C₁-C₆, linear and branched, substituted and unsubstituted alkyl radicals;
alkylamide groups, in which the alkyl is chosen from C₁-C₆, linear and branched, substituted and unsubstituted alkyl radicals;
alkylcarbamyl groups, in which the alkyl is chosen from C₁-C₆, linear and branched, substituted and unsubstituted alkyl radicals;
cyano groups;
nitro groups;
sulfonyl groups;
alkylsulfonyl groups in which the alkyl is chosen from C₁-C₆, linear and branched, substituted and unsubstituted alkyl radicals;
alkylsulfonylamido groups in which the alkyl is chosen from C₁-C₆, linear and branched, substituted and unsubstituted alkyl radicals; and
alkyl radicals, if the substituted radical is itself other than an alkyl radical.

It is to be noted that the alkyl parts of the groups mentioned above, may, themselves also, be substituted with at least one of the listed groups. However, in accordance with one embodiment of the present disclosure, the alkyl parts of the groups are not substituted.

As regards the cyclic and polycyclic radicals comprising at least one hetero atom, these radicals are 5- to 8-membered, such as 5- or 6-membered radicals. The at least one hetero atom is chosen from oxygen, nitrogen and sulfur. Furthermore, these cyclic and polycyclic radicals may be chosen from saturated, unsaturated, and aromatic radicals. Non-limiting examples that may be mentioned include pyrrolidine, piperidine, piperazine, pyrazole, imidazole, triazole, oxazole, thiazole, pyridine, pyrimidine, pyrazine and pyridazine rings.

According to one embodiment of the present disclosure, R₁ and R₆, which may be identical or different, may be chosen from hydrogen atoms, hydroxyl radicals, C₁-C₆ alkyl radicals, C₁-C₆ monohydroxyalkyl radicals, C₂-C₆ polyhydroxyalkyl radicals, C₁-C₆ alkoxy radicals, C₁-C₆ alkylcarbonyl radicals, amino radicals, amino radicals mono- and disubstituted with at least one radical chosen from C₁-C₆ alkyl radicals and C₁-C₆ hydroxyalkyl radicals; benzyl and phenyl radicals optionally substituted as disclosed above; nitro groups; and cyano groups.

In accordance with another embodiment of the present disclosure, the radicals R₂, R₃, R₄, and R₅, which may be identical or different, may be chosen from hydrogen atoms; hydroxyl radicals; thiol radicals; halogen atoms, such as chlorine and fluorine; C₁-C₆ alkyl radicals; C₁-C₆ monohydroxyalkyl radicals; C₂-C₆ polyhydroxyalkyl radicals; C₁-C₆ alkoxy radicals; C₁-C₆ alkylcarbonyl radicals; amino radicals; amino radicals mono- and disubstituted with at least one radical chosen from C₁-C₆ alkyl radicals and C₁-C₆ hydroxyalkyl radicals; benzyl and phenyl radicals optionally substituted as disclosed above; nitro groups; cyano groups; and heterocyclic groups.

According to one embodiment of the present disclosure, A is an oxygen atom. In this embodiment, at least one of the radicals R₁ and R₆ is a hydroxyl radical, and the other radical may be a hydrogen atom.

According to yet another embodiment of the present disclosure, the radicals R₂ and R₅ are not both a hydrogen atom. Moreover, in another embodiment, the compound of formula (I) is different from 2,5,7-trimethyltropolone, 7-(2-propyl)tropolone and hinokitiol.

Still according to this embodiment, at least one of the radicals R₃ and R₄ of formula (I) is chosen from C₆-C₁₂ aryl radicals which are unsubstituted or substituted with a hydroxyl radical; halogen atoms; C₁-C₄ alkoxy radicals; amino radicals; amino radicals substituted with one or two C₁-C₄ alkyl radicals, which may be identical or different; and thiol radicals. The other radical of R₃ and R₄ may be a hydrogen atom.

Among the compounds that may be used according to this first embodiment, non-limiting mention may be made of 5-(4′-N,N-dimethylamino)phenyltropolone.

In accordance with yet another embodiment of the present disclosure, the compound of formula (I) may be such that A is chosen from C(R₇)(R₈) radicals in which R₇ and R₈, which may be identical or different, are electron-withdrawing monovalent groups.

By way of non-limiting example, the electron-withdrawing monovalent groups may be chosen from cyano radicals; —COOX radicals wherein X is chosen from a hydrogen atom, alkali metals, alkaline-earth metals, and linear and branched C₁-C₆ alkyl radicals; halogen atoms such as, for example, chlorine and fluorine; —NO₂ radicals; —SO₃X′ radicals wherein X′ is chosen from a hydrogen atom, alkali metals and alkaline-earth metals, for instance sodium, potassium, magnesium or calcium.

According to one aspect of this embodiment, the compound of formula (I) is such that R₇ and R₈ are chosen from cyano radicals.

Further according to this embodiment, at least one of the radicals R₃ and R₄ may be chosen from C₆-C₁₂ aryl radicals, which may be unsubstituted or substituted with a hydroxyl radical; halogen atoms; C₁-C₄ alkoxy radicals; amino radicals; amino radicals substituted with at least one C₁-C₄ alkyl radicals; and thiol radicals. The other radical may be a hydrogen atom.

Still in the context of this embodiment, at least one of the radicals R₁, R₂, R₅ and R₆ is a hydrogen atom. For example, the radicals R₁, R₂, R₅ and R₆ may all be a hydrogen atom.

Among the compounds that may be used within the context of this embodiment, non-limiting mention may be made, inter alia, of 8,8-dicyano-3-(4′-methoxy)phenylheptafulvene; 8,8-dicyano-3-(4′-N,N-dimethylamino)phenylheptafulvene; 8,8-dicyano-3-phenylheptafulvene, or mixtures thereof.

In general, the compound of formula (I) is in the form of a dye. Thus, the compound is present in an amount such that it is in a form that is soluble in the medium of the composition. For example, at least 50% by weight of the dye can be soluble in the medium, such as at least 90% by weight. For example, compound (I) can be present in the composition according to the present disclosure in an amount ranging from 0.1% to 20% by weight, relative to the weight of the composition, such as from 0.5% to 5% by weight, relative to the weight of the composition.

The composition may further comprise other ingredients that are conventional in the field of dye compositions for dyeing human keratin fibers, such as the hair. Thus, the composition may comprise at least one fluorescent or non-fluorescent direct dye, other than the direct dye that has just been described. The at least one additional direct dye may be chosen from nonionic, cationic, anionic, amphoteric and zwitterionic species, such as nonionic and cationic species.

Non-limiting examples that may be mentioned include nitrobenzene direct dyes, acridine, acridone, anthranthrone, anthrapyrimidine, anthraquinone, azine, azo, azomethine, benzathrone, benzimidazole, benzimidazolone, benzindole, benzoxazole, benzopyran, benzothiazole, benzoquinone, bisazine, bisisoindoline, carboxanilide, coumarin, cyanin (for instance azacarbocyanin, diazacarbocyanin, diazahemicyanin, hemicyanin and tetraazacarbocyanin), diazin, diketopyrrolopyrrole, dioxazine, diphenylamine, diphenylmethane, dithiazine, flavanthrone, flavone, fluorindine, formazan, hydrazone, hydroxy ketone, indamine, indanthrone, indigoid, indophenol, indoaniline, isoindoline, isoindoline, isoindolinone, isoviolanthrone, lactone, methine, naphthalimide, naphthanilide, naphtholactam, naphthoquinone, nitro, oxadiazole, oxazine, perilone, perinone, perylene, phenazine, phenothiazine, phthalocyanin, polyene/carotenoid, porphyrin, pyranthrone, pyrazolanthrone, pyrazolone, pyrimidinoanthrone, pyronine, quinacridone, quinoline, quinophthalone, squarane, stilbene, tetrazolium, thiazine, thioindigo, thiopyronine, triarylmethane and xanthene dyes.

Fluorescent direct dyes chosen from the cationic fluorescent compounds of the family of azo, azomethine, methine, naphthalimide, cationic or non-cationic coumarin, xanthenodiquinolizine, azaxanthene, naphtholactam, azlactone, (di)oxazine and thiazine compounds, alone or as mixtures, are also suitable for use.

If the composition does comprise at least one additional fluorescent or non-fluorescent direct dye, the dye(s) may be present in the composition in an amount ranging from 0.0005% to 12% by weight, relative to the weight of the composition, such as from 0.005% to 6% by weight, relative to the weight of the composition.

The composition according to the present disclosure may also comprise at least one oxidation base optionally combined with at least one coupler. The at least one oxidation base may be chosen from the oxidation compounds that are conventional in the field of dyeing.

Non-limiting examples that may be mentioned, inter alia, include o-phenylenediamines, p-phenylenediamines, double bases, o-aminophenols, p-aminophenols and heterocyclic bases, the acid addition salts thereof, and also mixtures thereof.

If the composition comprises at least one oxidation base, the oxidation base(s) can be present in an amount ranging from 0.0005% to 12% by weight, relative to the weight of the composition, such as from 0.005% to 8% by weight, relative to the weight of the composition.

Among the couplers that may be used in combination with at least one oxidation base as disclosed herein, non-limiting mention may be made, for example, of m-aminophenols, m-phenylenediamines, m-diphenols, naphthols and heterocyclic couplers, acid addition salts thereof, and also mixtures thereof.

In the case where at least one coupler is present in the composition, it may be present in an amount ranging from 0.0001% to 10% by weight, relative to the weight of the composition, such as from 0.005% to 5% by weight, relative to the weight of the composition.

In general, the acid addition salts of the oxidation bases and couplers can be chosen from the hydrochlorides, hydrobromides, sulfates, tartrates, lactates and acetates.

The composition according to the present disclosure may also comprise various conventionally used adjuvants, such as anionic, cationic, nonionic, amphoteric and zwitterionic surfactants, and mixtures thereof; anionic, cationic, nonionic, amphoteric and zwitterionic polymers; mineral thickeners; antioxidants; penetrating agents; fragrances; buffers; dispersing agents; conditioners, for instance cations, volatile and non-volatile, modified and unmodified silicones; cationic amphiphilic polymers; film-forming agents; ceramides; vitamins and/or provitamins; preserving agents; stabilizers; opacifiers and matting agents, for instance titanium dioxide; mineral fillers, for instance clays; silicas, such as fumed silicas of hydrophilic or hydrophobic nature; polymeric binders such as vinylpyrrolidone; sunscreens; etc.

The adjuvants mentioned above can be present in an amount for each of them ranging from 0.01% to 20% by weight, relative to the weight of the composition.

The medium of the composition is a cosmetically acceptable medium. The cosmetically acceptable medium may consist of water, or comprise a mixture of water and at least one organic solvent.

Non-limiting examples among the common organic solvents that may be mentioned include linear and branched, for example saturated, monoalcohols comprising from 2 to 10 carbon atoms, such as ethyl alcohol or isopropyl alcohol; aromatic alcohols such as benzyl alcohol or phenylethyl alcohol; polyols or polyol ethers, for instance ethylene glycol monomethyl, monoethyl or monobutyl ether, propylene glycol or ethers thereof, for instance propylene glycol monomethyl ether, butylene glycol, dipropylene glycol, hexylene glycol (2-methyl-2,4-pentanediol), neopentyl glycol and 3-methyl-1,5-pentanediol; and also diethylene glycol alkyl ethers, such as C₁-C₄, for instance diethylene glycol monoethyl ether or monobutyl ether, alone or as a mixture.

When they are present, the organic solvents described above can be present in an amount ranging from 1% to 40% by weight, for instance, ranging from 5% to 30% by weight, relative to the total weight of the composition.

The pH of the composition according to the present disclosure can range from 3 to 12, such as from 5 to 11. The pH may be adjusted to the desired value using acidifying or basifying agents.

Among the acidifying agents that may be used, non-limiting examples that may be mentioned include mineral or organic acids, for instance hydrochloric acid, orthophosphoric acid, sulfuric acid and acetic acid.

Among the basifying agents that can be used, non-limiting examples that may be mentioned include aqueous ammonia, alkali metal carbonates, alkanolamines such as monoethanolamine, diethanolamine or triethanolamine, and derivatives thereof, sodium hydroxide, potassium hydroxide and the compounds of formula (II) below: 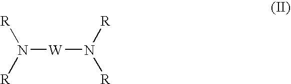
wherein W is a propylene residue optionally substituted with a radical chosen from hydroxyl groups and C₁-C₆ alkyl radicals; the radicals R, which may be identical or different, are chosen from hydrogen atoms and C₁-C₆ alkyl and C₁-C₆ hydroxyalkyl radicals.

It should be noted that the composition may be in the form of a thickened or non-thickened lotion, a cream, a gel, a mousse, a spray or any other form that is suitable for the subsequent application of this composition.

Another aspect of the present disclosure is that of a ready-to-use composition comprising a composition comprising at least one compound of formula (I) as disclosed herein, and at least one oxidizing agent.

For example, the oxidizing agent can be chosen from hydrogen peroxide, alkali metal and alkaline-earth metal peroxides, for instance sodium, potassium and magnesium peroxide; urea peroxide, alkali metal bromates and ferricyanides, and persalts such as perborates and persulfates of alkali metals or of alkaline-earth metals, for instance sodium, potassium or magnesium, alone or as mixtures. The at least one oxidizing agent can also be chosen from enzymes such as peroxidases and two-electron and four-electron oxidoreductases.

The at least one oxidizing agent can be present in an amount ranging from 0.001% to 20% by weight, relative to the weight of the ready-to-use composition, such as from 0.1% to 12% by weight, relative to the weight of the ready-to-use composition.

The pH of the ready-to-use composition can range from 4 to 12, such as from 6 to 11. The pH of the ready-to-use composition can be adjusted to the desired value using acidifying or basifying agents such as those described previously.

The ready-to-use composition results, for example, from mixing the composition comprising at least one compound of formula (I) according to the present disclosure with a composition comprising at least one oxidizing agent (known as an oxidizing composition). In this case, the mixture is prepared before the application of the ready-to-use composition to the fibers to be treated.

The medium of the oxidizing composition is one that is suitable for dyeing, for example, water or a mixture of water and at least one organic solvent.

Finally, the oxidizing composition may also comprise at least one additive that is common in the field, for instance surfactants, thickeners, antioxidants, fragrances, dispersants, conditioners, sequestering agents, preserving agents, etc.

It should be pointed out that the lists of solvents and additives, and similarly the contents thereof, indicated in the context of the description of the composition comprising at least one compound of formula (I) according to the present disclosure remain valid and reference may be made thereto.

Another subject of the invention is a process for dyeing keratin fibers, such as human keratin fibers, for example, the hair, in which the composition that has just been detailed is applied to wet or dry keratin fibers.

For example, the present disclosure relates to a process for dyeing human keratin fibers, such as the hair, comprising,
applying to wet or dry fibers a composition comprising at least one compound of formula (I) and leaving the composition on the fibers for a period of time that is sufficient to develop coloration,
optionally rinsing the fibers,
optionally washing and rinsing the fibers,
and drying or leaving to dry the fibers.

In accordance with one aspect of the present disclosure, the process is performed without rinsing out the composition. Thus, after the composition is applied to the fibers, they are dried or left to dry. This procedure may be suitable, for example, in the case where the composition does not comprise any oxidizing agent, oxidation base or coupler.

In accordance with another aspect of the present disclosure, the process is performed with at least one rinsing step.

According to an embodiment of the process, the composition applied to the fibers does not comprise any oxidizing agent. This embodiment can be suitable when, for example, the composition comprises only at least one compound of formula (I) and optionally at least one fluorescent or non-fluorescent direct dye.

According to another embodiment of the process, the composition applied to the fibers is a ready-to-use composition as described above. This embodiment of the process is suitable irrespective of the composition according to the present disclosure that is used, whether or not this composition comprises, besides the at least one compound of formula (I), a fluorescent or non-fluorescent direct dye, at least one oxidation base and optionally at least one coupler. In accordance with this embodiment, a first possibility comprises applying the ready-to-use composition, obtained by extemporaneous mixing before application to the fibers, of the composition comprising at least one compound of formula (I) as disclosed herein, and of an oxidizing composition. According to this possibility, it may be desired to separately store the composition comprising at least one compound of formula (I) according to the present disclosure, and separately store the oxidizing composition, before use.

A second possibility comprises successively applying, in any order, the composition comprising at least one compound of formula (I) according to the present disclosure, followed by applying the oxidizing composition, or alternatively comprises applying the two compositions simultaneously.

The period of time required to develop the coloration can range from 1 to 60 minutes, such as from 5 to 45 minutes.

Moreover, the application of the dye composition to the wet or dry fibers, and period time for developing of coloration, can be performed at a temperature ranging from 15° C. to 220° C., for instance at a temperature ranging from 15° C. to 40° C. Once the development of coloration is complete, the fibers may optionally be rinsed, optionally washed with a shampoo and then rinsed, and finally dried or left to dry, for example at a temperature ranging from 20° C. to 120° C.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the invention in a non-limiting manner.

### EXAMPLES

The following formulations were prepared:

| Starting material | Gram % |
|---|---|
| Dye of formula (I) | 0.25 |
| Ethanol | Amount required to dissolve the |
| | compound of formula (I) |
| Deionized water | qs 50 |

### Example 1

### 8,8-Dicyano-3-(4′-methoxy)phenylheptafulvene of formula

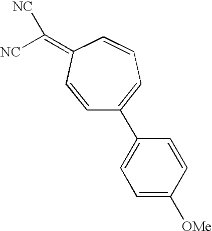

### Example 2

### 8,8-Dicyano-3-(4′-N,N-dimethylamino)phenylheptafulvene of formula

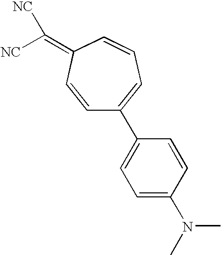

### Example 3

### 8,8-Dicyano-3-phenylheptafulvene of formula

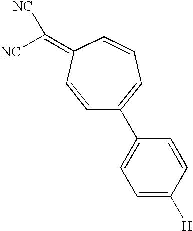

### Example 4

### 5-(4′-N,N-Dimethylamino)phenyltropolone of formula

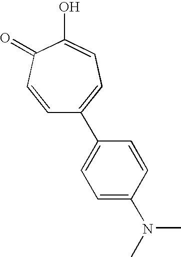

For each example, 10 g of each of the above formulations were applied for 30 minutes at room temperature to 1 g locks of natural hair containing 90% white hairs.

After the leave-in time ended, the locks were rinsed with water and dried.

The colorations below were obtained:

| Examples | Color of the hair after dyeing |
|---|---|
| Example 1 | Red-orange |
| Example 2 | Violet-black |
| Example 3 | Red-orange |
| Example 4 | Yellow |

1. A composition comprising, in a medium that is suitable for dyeing keratin fibers at least one compound of formula (I): 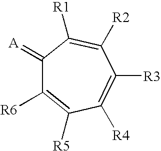
wherein:
R₁ and R₆, which may be identical or different, are chosen from hydrogen atoms;
alkyl radicals, aryl radicals, alkylaryl radicals and arylalkyl radicals, wherein the alkyl is chosen from C₁-C₆, linear and branched, substituted and unsubstituted alkyl radicals, and the aryl is chosen from C₆-C₁₂, substituted and unsubstituted aryl radicals; halogen atoms; C₁-C₆ alkoxy radicals; amino radicals; amino radicals substituted with at least one radical, which may be identical or different, chosen from linear and branched, substituted and unsubstituted C₁-C₆ alkyl radicals; hydroxyl radicals; —COOX radicals, wherein X is chosen from a hydrogen atom, alkali metals, alkaline-earth metals, and linear and branched, substituted and unsubstituted C₁-C₆ alkyl radicals; alkylcarbonyl radicals in which the alkyl is chosen from C₁-C₆, linear and branched, substituted and unsubstituted alkyl radicals; —SO₃X′ radicals wherein X′ is chosen from a hydrogen atom and alkali metals and alkaline-earth metals;
R₂, R₃, R₄ and R₅, which may be identical or different, are chosen from hydrogen atoms; linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals; arylalkyl radicals in which the alkyl is chosen from C₁-C₆, linear and branched, substituted and unsubstituted alkyl radicals, and the aryl is chosen from C₆-C₁₂ aryl radicals; substituted and unsubstituted cyclic and polycyclic radicals comprising at least one hetero atom;
A is chosen from oxygen and sulfur atoms, and C(R₇)(R₈) groups in which R₇ and R₈, which may be identical or different, are chosen from electron-withdrawing monovalent groups.

2. The composition according to claim 1, wherein the keratin fibers are human keratin fibers.

3. The composition according to claim 1, wherein the substituted radicals of R₁, R₂, R₃, R₄, R₅, and R₆ bear at least one group chosen from:
a hydroxyl groups;
n halogen atoms;
C₁-C₆ alkoxy groups;
a monohydroxyalkoxy groups in which the alkyl is chosen from C₁-C₆, linear and branched, substituted and unsubstituted alkyl radicals;
a polyhydroxyalkoxy groups, in which the alkyl is chosen from C₂-C₆, linear and branched, substituted and unsubstituted alkyl radicals;
amino groups, which are unsubstituted or substituted with at least one radical chosen from linear and branched, substituted and unsubstituted C₁-C₆ alkyl radicals;
thiol groups;
alkylthio groups in which the alkyl is chosen from C₁-C₆, linear and branched, substituted and unsubstituted alkyl radicals;
carboxylic groups in acid or salified form;
alkylcarbonyl groups in which the alkyl is chosen from C₁-C₆ linear and branched, substituted and unsubstituted alkyl radicals;
alkoxycarbonyl groups in which the alkyl is chosen from C₁-C₆ linear and branched, substituted and unsubstituted alkyl radicals;
alkylamide groups, wherein the alkyl is chosen from C₁-C₆, linear and branched, substituted and unsubstituted alkyl radicals;
alkylcarbamyl groups, the alkyl of which is chosen from C₁-C₆ linear and branched, substituted and unsubstituted alkyl radicals;
cyano groups;
nitro groups;
sulfonyl groups;
alkylsulfonyl groups in which the alkyl is chosen from C₁-C₆ linear and branched, substituted and unsubstituted alkyl radicals;
alkylsulfonylamido groups in which the alkyl is chosen from C₁-C₆ linear and branched, substituted and unsubstituted alkyl radicals; and
alkyl radicals, if the radical to be substituted is itself other than an alkyl radical.

4. The composition according to claim 3, wherein the halogens are chosen from chlorine and fluorine.

5. The composition according to claim 3, wherein the carboxylic groups are in salified form with an alkali metal or a substituted or unsubstituted ammonium.

6. The composition according to claim 1, wherein A is chosen from C(R₇)(R₈) radicals in which R₇ and R₈, which may be identical or different, are electron-withdrawing monovalent groups chosen from cyano radicals; —COOX radicals wherein X is chosen from a hydrogen atom, alkali metals, alkaline-earth metals, and linear and branched C₁-C₆ alkyl radicals; halogen atoms; —NO₂ radicals; —SO₃X′ radicals wherein X′ is chosen from a hydrogen atom, alkali metals and alkaline-earth metals.

7. The composition according to claim 1, wherein the radicals R₂, R₃, R₄, and R₅ are chosen from saturated, unsaturated, and aromatic, optionally substituted, 5- to 8-membered cyclic and polycyclic radicals comprising at least one hetero atom chosen from oxygen, nitrogen and sulfur atoms.

8. The composition according to claim 7, wherein R₂, R₃, R₄, and R₅ are chosen from saturated, unsaturated, and aromatic, optionally substituted, 5- or 6-membered cyclic and polycyclic radicals comprising at least one hetero atom chosen from oxygen, nitrogen and sulfur atoms.

9. The composition according to claim 3, wherein R₁ and R₆, which may be identical or different, are chosen from hydrogen atoms, hydroxyl radicals, C₁-C₆ alkyl radicals, C₁-C₆ monohydroxyalkyl radicals, C₂-C₆ polyhydroxyalkyl radicals, C₁-C₆ alkoxy radicals, C₁-C₆ alkylcarbonyl radicals, amino radicals, amino radicals mono- and disubstituted with at least one radical chosen from C₁-C₆ alkyl radicals and C₁-C₆ hydroxyalkyl radicals; optionally substituted benzyl and phenyl radicals; nitro groups; and cyano groups.

10. The composition according to claim 3, wherein R₂, R₃, R₄, and R₅, which may be identical or different, are chosen from hydrogen atoms; hydroxyl radicals; thiol radicals; halogen atoms; C₁-C₆ alkyl radicals; C₁-C₆ monohydroxyalkyl radicals; C₂-C₆ polyhydroxyalkyl radicals; C₁-C₆ alkoxy radicals; C₁-C₆ alkylcarbonyl radicals; amino radicals; amino radicals mono- and disubstituted with at least one radical chosen from C₁-C₆ alkyl radicals and C₁-C₆ hydroxyalkyl radicals; optionally substituted benzyl and phenyl radicals; nitro groups; cyano groups; and heterocyclic groups.

11. The composition according to claim 10, wherein the halogen atoms are chosen from chlorine and fluorine.

12. The composition according to claim 1, wherein A is an oxygen atom and at least one of R₁ or R₆ is a hydroxyl radical.

13. The composition according to claim 12, wherein at least one of R₃ and R₄ is a radical chosen from C₆-C₁₂ aryl radicals which are unsubstituted or substituted with a hydroxyl radical; halogen atoms; C₁-C₄ alkoxy radicals; amino radicals; amino radicals substituted with one or two C₁-C₄ alkyl radicals; and thiol radicals; and wherein the other of R₃ or R₄ is a hydrogen atom.

14. The composition according to claim 1, wherein the at least one compound of formula (I) is 5-(4′-N,N-dimethylamino)phenyltropolone.

15. The composition according to claim 1, wherein A is chosen from C(R₇)(R₈) radicals, wherein R₇ and R₈ are cyano radicals.

16. The composition according to claim 15, wherein one of R₃ or R₄ is chosen from C₆-C₁₂ aryl radicals which are unsubstituted or substituted with a hydroxyl radical; halogen atoms; C₁-C₄ alkoxy radicals; amino radicals; amino radicals substituted with at least one C₁-C₄ alkyl radical, which may be identical or different; and thiol radicals.

17. The composition according to claim 16, wherein R₁, R₂, R₅ and R₆ are hydrogen atoms.

18. The composition according to claim 1, wherein the at least one compound of formula (I) is chosen from 8,8-dicyano-3-(4′-methoxy)phenylheptafulvene; 8,8-dicyano-3-(4′-N,N-dimethylamino)phenylheptafulvene; and 8,8-dicyano-3-phenylheptafulvene.

19. The composition according to claim 1, wherein the at least one compound of formula (I) is present in the composition in an amount ranging from 0.1% to 20% by weight, relative to the weight of the composition.

20. The composition according to claim 19, wherein the at least one compound of formula (I) is present in the composition in an amount ranging from 0.5% to 5% by weight, relative to the weight of the composition.

21. The composition according to claim 1, further comprising at least one additional direct dye chosen from fluorescent and non-fluorescent direct dyes.

22. The composition according to claim 21, wherein the at least one additional direct dye is chosen from nitrobenzene dyes, acridine, acridone, anthranthrone, anthrapyrimidine, anthraquinone, azine, azo, azomethine, benzathrone, benzimidazole, benzimidazolone, benzindole, benzoxazole, benzopyran, benzothiazole, benzoquinone, bisazine, bis-isoindoline, carboxanilide, coumarin, cyanin, diazin, diketopyrrolopyrrole, dioxazine, diphenylamine, diphenylmethane, dithiazine, flavanthrone, flavone, fluorindine, formazan, hydrazone, hydroxy ketone, indamine, indanthrone, indigoid, indophenol, indoaniline, isoindoline, isoindoline, isoindolinone, isoviolanthrone, lactone, methine, naphthalimide, naphthanilide, naphtholactam, naphthoquinone, nitro, oxadiazole, oxazine, perilone, perinone, perylene, phenazine, phenothiazine, phthalocyanin, polyene/carotenoid, porphyrin, pyranthrone, pyrazolanthrone, pyrazolone, pyrimidinoanthrone, pyronine, quinacridone, quinoline, quinophthalone, squarane, stilbene, tetrazolium, thiazine, thioindigo, thiopyronine, triarylmethane and xanthene dyes.

23. The composition according to claim 21, wherein the at least one fluorescent direct dye is chosen from cationic fluorescent compounds of the family of azo, azomethine, methine, naphthalimide, cationic coumarin, xanthenodiquinolizine, azaxanthene, naphtholactam, azlactone, (di)oxazine and thiazine compounds.

24. The composition according to claim 21, wherein the at least one additional direct dye is present in an amount ranging from 0.0005% to 12% by weight, relative to the weight of the composition.

25. The composition according to claim 24, wherein the at least one additional direct dye is present in an amount ranging from 0.005% to 6% by weight, relative to the weight of the composition.

26. The composition according to claim 1, further comprising at least one oxidation base optionally combined with at least one coupler.

27. The composition according to claim 26, wherein the at least one oxidation base is chosen from o-phenylenediamines, p-phenylenediamines, double bases, o-aminophenols, p-aminophenols, heterocyclic bases, and the acid addition salts thereof.

28. The composition according to claim 27, wherein the at least one oxidation base is present in a total amount ranging from 0.0005% to 12% by weight, relative to the weight of the composition.

29. The composition according to claim 28, wherein the at least one oxidation base is present in a total amount ranging from 0.005% to 8% by weight, relative to the weight of the composition.

30. The composition according to claim 26, wherein the at least one coupler is chosen from m-aminophenols, m-phenylenediamines, m-diphenols, naphthols, heterocyclic couplers, and the acid addition salts thereof.

31. The composition according to claim 26, wherein the at least one coupler is present in a total amount ranging from 0.0001% to 10% by weight, relative to the weight of the composition.

32. The composition according to claim 31, wherein the at least one coupler is present in a total amount ranging from 0.005% to 5% by weight, relative to the weight of the composition.

33. A ready-to-use dye composition, comprising, in a medium that is suitable for dyeing keratin fibers, at least one oxidizing agent, and
at least one compound of formula (I): 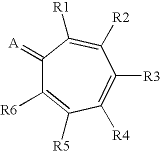
wherein:
R₁ and R₆, which may be identical or different, are chosen from hydrogen atoms; alkyl radicals, aryl radicals, alkylaryl radicals and arylalkyl radicals, in which the alkyl is chosen from C₁-C₆, linear and branched, substituted and unsubstituted alkyl radicals, and the aryl is chosen from C₆-C₁₂, substituted and unsubstituted aryl radicals; halogen atoms; C₁-C₆ alkoxy radicals; amino radicals; amino radicals substituted with at least one radical, which may be identical or different, and is chosen from linear and branched, substituted and unsubstituted C₁-C₆ alkyl radicals; hydroxyl radicals; —COOX radicals, wherein X is chosen from a hydrogen atom, alkali metals, alkaline-earth metals, and linear and branched, substituted and unsubstituted C₁-C₆ alkyl radicals; alkylcarbonyl radicals in which the alkyl is chosen from C₁-C₆, linear and branched, substituted and unsubstituted alkyl radicals; —SO₃X′ radicals wherein X′ is chosen from a hydrogen atom and alkali metals and alkaline-earth metals;
R₂, R₃, R₄ and R₅, which may be identical or different, are chosen from hydrogen atoms; linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals; arylalkyl radicals in which the alkyl is chosen from C₁-C₆, linear and branched, substituted and unsubstituted alkyl radicals, and the aryl is chosen from C₆-C₁₂ aryl radicals; substituted and unsubstituted cyclic and polycyclic radicals comprising at least one hetero atom;
A is chosen from oxygen and sulfur atoms, and C(R₇)(R₈) groups in which R₇ and R₈, which may be identical or different, are chosen from electron-withdrawing monovalent groups.

34. The ready-to-use dye composition according to claim 33, wherein the at least one oxidizing agent is chosen from hydrogen peroxide, urea peroxide, alkali metal bromates and ferricyanides, persalts, and enzymes, alone or as mixtures.

35. The ready-to-use dye composition according to claim 34, wherein the at least one persalt is chosen from perborates and persulfates.

36. A process for dyeing keratin fibers, comprising applying to wet or dry keratin fibers a composition comprising, in a medium that is suitable for dyeing keratin fibers, at least one compound of formula (I): 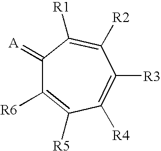
wherein:
R₁ and R₆, which may be identical or different, are chosen from hydrogen atoms; alkyl radicals, aryl radicals, alkylaryl radicals and arylalkyl radicals, in which the alkyl is chosen from C₁-C₆, linear and branched, substituted and unsubstituted alkyl radicals, and the aryl is chosen from C₆-C₁₂, substituted and unsubstituted aryl radicals; halogen atoms; C₁-C₆ alkoxy radicals; amino radicals; amino radicals substituted with at least one radical, which may be identical or different, chosen from linear and branched, substituted and unsubstituted C₁-C₆ alkyl radicals; hydroxyl radicals; —COOX radicals, wherein X is chosen from a hydrogen atom, alkali metals, alkaline-earth metals, and linear and branched, substituted and unsubstituted C₁-C₆ alkyl radicals; alkylcarbonyl radicals in which the alkyl is chosen from C₁-C₆, linear and branched, substituted and unsubstituted alkyl radicals; —SO₃X′ radicals wherein X′ is chosen from a hydrogen atom and alkali metals and alkaline-earth metals;
R₂, R₃, R₄ and R₅, which may be identical or different, are chosen from hydrogen atoms; linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals; arylalkyl radicals in which the alkyl is chosen from C₁-C₆, linear and branched, substituted and unsubstituted alkyl radicals, and the aryl is chosen from C₆-C₁₂ aryl radicals; substituted and unsubstituted cyclic and polycyclic radicals comprising at least one hetero atom;
A is chosen from oxygen and sulfur atoms, and C(R₇)(R₈) groups in which R₇ and R₈, which may be identical or different, are chosen from electron-withdrawing monovalent groups.

37. The process according to claim 36, wherein the keratin fibers are human keratin fibers.

38. The process according to claim 36, wherein, after the composition is applied to the fibers, they are dried or are left to dry.

39. A process for dyeing human keratin fibers, wherein:
wet or dry keratin fibers are placed in contact with a composition comprising, in a medium that is suitable for dyeing keratin fibers,
at least one compound of formula (I): 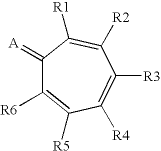
wherein:
R₁ and R₆, which may be identical or different, are chosen from hydrogen atoms; alkyl radicals, aryl radicals, alkylaryl radicals and arylalkyl radicals, in which the alkyl is chosen from C₁-C₆, linear and branched, substituted and unsubstituted alkyl radicals, and the aryl is chosen from C₆-C₁₂, substituted and unsubstituted aryl radicals; halogen atoms; C₁-C₆ alkoxy radicals; amino radicals; amino radicals substituted with at least one radical, which may be identical or different, chosen from linear and branched, substituted and unsubstituted C₁-C₆ alkyl radicals; hydroxyl radicals; —COOX radicals, wherein X is chosen from a hydrogen atom, alkali metals, alkaline-earth metals, and linear and branched, substituted and unsubstituted C₁-C₆ alkyl radicals; alkylcarbonyl radicals in which the alkyl is chosen from C₁-C₆, linear and branched, substituted and unsubstituted alkyl radicals; —SO₃X′ radicals wherein X′ is chosen from a hydrogen atom and alkali metals and alkaline-earth metals;
R₂, R₃, R₄ and R₅, which may be identical or different, are chosen from hydrogen atoms; linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals; arylalkyl radicals in which the alkyl is chosen from C₁-C₆, linear and branched, substituted and unsubstituted alkyl radicals, and the aryl radicals are chosen from C₆-C₁₂ radicals; substituted and unsubstituted cyclic and polycyclic radicals comprising at least one hetero atom;
A is chosen from oxygen and sulfur atoms, and C(R₇)(R₈) groups in which R₇ and R₈, which may be identical or different, are chosen from electron-withdrawing monovalent groups;
wherein the fibers are optionally rinsed, and/or the fibers are optionally washed and rinsed, and/or the fibers are dried or are left to dry.

40. A multi-compartment kit for dyeing keratin fibers, comprising
at least one first compartment comprising, in a medium that is suitable for dyeing keratin fibers, at least one compound of formula (I): 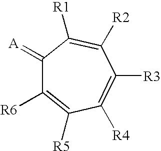
wherein:
R₁ and R₆, which may be identical or different, are chosen from hydrogen atoms; alkyl radicals, aryl radicals, alkylaryl radicals and arylalkyl radicals, in which the alkyl is chosen from C₁-C₆, linear and branched, substituted and unsubstituted alkyl radicals, and the aryl is chosen from C₆-C₁₂, substituted and unsubstituted aryl radicals; halogen atoms; C₁-C₆ alkoxy radicals; amino radicals; amino radicals substituted with at least one radical, which may be identical or different, chosen from linear and branched, substituted and unsubstituted C₁-C₆ alkyl radicals; hydroxyl radicals; —COOX radicals, wherein X is chosen from a hydrogen atom, alkali metals, alkaline-earth metals, and linear and branched, substituted and unsubstituted C₁-C₆ alkyl radicals; alkylcarbonyl radicals in which the alkyl is chosen from C₁-C₆, linear and branched, substituted and unsubstituted radicals; —SO₃X′ radicals wherein X′ is chosen from a hydrogen atom and alkali metals and alkaline-earth metals;
R₂, R₃, R₄ and R₅, which may be identical or different, are chosen from hydrogen atoms; linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals; arylalkyl radicals in which the alkyl is chosen from C₁-C₆, linear and branched, substituted and unsubstituted radicals, and the aryl is chosen from C₆-C₁₂ radicals; substituted and unsubstituted cyclic and polycyclic radicals comprising at least one hetero atom;
A is chosen from oxygen and sulfur atoms, and C(R₇)(R₈) groups in which R₇ and R₈, which may be identical or different, are chosen from electron-withdrawing monovalent groups, and
at least one second compartment comprising a composition comprising at least one oxidizing agent.

41. A process for dyeing keratin fibers in natural and chromatic shades, comprising applying to wet or dry keratin fibers a composition comprising, in a medium that is suitable for dyeing keratin fibers, at least one compound of formula (I): 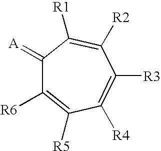
wherein:
R₁ and R₆, which may be identical or different, are chosen from hydrogen atoms; alkyl radicals, aryl radicals, alkylaryl radicals and arylalkyl radicals, in which the alkyl is chosen from C₁-C₆, linear and branched, substituted and unsubstituted alkyl radicals, and the aryl is chosen from C₆-C₁₂, substituted and unsubstituted aryl radicals; halogen atoms; C₁-C₆ alkoxy radicals; amino radicals; amino radicals substituted with at least one radical, which may be identical or different, chosen from linear and branched, substituted and unsubstituted C₁-C₆ alkyl radicals; hydroxyl radicals; —COOX radicals, wherein X is chosen from a hydrogen atom, alkali metals, alkaline-earth metals, and linear and branched, substituted and unsubstituted C₁-C₆ alkyl radicals; alkylcarbonyl radicals in which the alkyl is chosen from C₁-C₆, linear and branched, substituted and unsubstituted alkyl radicals; —SO₃X′ radicals wherein X′ is chosen from a hydrogen atom and alkali metals and alkaline-earth metals;
R₂, R₃, R₄ and R₅, which may be identical or different, are chosen from hydrogen atoms; linear and branched, substituted and unsubstituted C₁-C₆ alkyl radicals; hydroxyl radicals; C₁-C₆ alkoxy radicals; thiol radicals; C₁-C₆ alkylthio radicals; amino radicals; amino radicals substituted with at least one radical, which may be identical or different, chosen from linear and branched, substituted and unsubstituted C₁-C₆ alkyl radicals; substituted and unsubstituted C₆-C₁₂ aryl radicals; arylalkyl radicals in which the alkyl is chosen from C₁-C₆, linear and branched, substituted and unsubstituted alkyl radicals, and the aryl is chosen from C₆-C₁₂ aryl radicals; substituted and unsubstituted cyclic and polycyclic radicals comprising at least one hetero atom;
A is chosen from oxygen and sulfur atoms, and C(R₇)(R₈) groups in which R₇ and R₈, which may be identical or different, are chosen from electron-withdrawing monovalent groups,
wherein the at least one compound of formula (I) is present in an effective amount such that, when dyed, the keratin fibers exhibit natural and chromatic shades.
